(19) United States
(12) Patent Application Publication
(10) Pub. No.: US 2026/0125355 A1
(43) Pub. Date: May 7, 2026

(54) COMPOSITION CONTAINING FLUORINE-CONTAINING MONOMER AND FLUORIDE IONS, METHOD FOR PRODUCING FLUORINE-CONTAINING MONOMER HAVING REDUCED FLUORIDE ION CONTENT, AND METHOD FOR PURIFYING FLUORINE-CONTAINING MONOMER

(71) Applicant: DAIKIN INDUSTRIES, LTD., Osaka (JP)

(72) Inventors: Daichi MARI, Osaka (JP); Noriyuki SHINOKI, Osaka (JP); Tadashi INO, Osaka (JP)

(73) Assignee: DAIKIN INDUSTRIES, LTD., Osaka (JP)

(21) Appl. No.: 19/439,710

(22) Filed: Jan. 5, 2026

Related U.S. Application Data
(63) Continuation of application No. PCT/JP2024/023756, filed on Jul. 1, 2024.

(30) Foreign Application Priority Data
Jul. 11, 2023 (JP) 2023-114063

(51) Int. Cl. C07D 317/42; C07C 43/17

(52) U.S. Cl. CPC C07D 317/42; C07C 43/17

(57) ABSTRACT

The present disclosure aims to provide a composition comprising a fluorine-containing monomer and fluoride ions wherein the amount of fluoride ions mixed in the fluorine-containing monomer is small, a method for producing a fluorine-containing monomer having a further reduced fluoride ion content from a fluorine-containing monomer contaminated with fluoride ions, a method for purifying a fluorine-containing monomer contaminated with fluoride ions, and the like. The present disclosure relates to a composition comprising a fluorine-containing monomer (M) and fluoride ions, wherein the fluoride ion content is 0.01 to 1000 ppm by mass based on the mass of the composition, and the fluorine-containing monomer (M) is at least one monomer selected from the group consisting of a compound represented by formula (M1), a compound represented by formula (M2), and a compound represented by formula (M3):

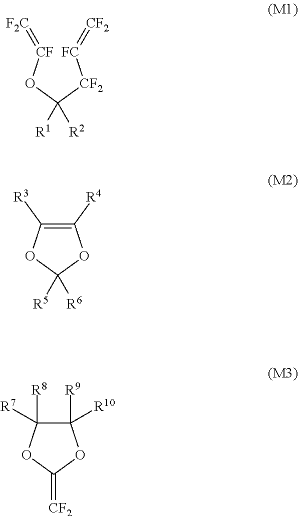

wherein R¹ to R¹⁰ each independently represent a fluorine atom, a perfluoroalkyl group, or a perfluoroalkoxy group.

### TECHNICAL FIELD

The present disclosure relates to a composition containing a fluorine-containing monomer and fluoride ions, a method for producing a fluorine-containing monomer having a reduced fluoride ion content, a method for purifying a fluorine-containing monomer, and the like.

### BACKGROUND ART

Specific fluorine-containing monomers are used as starting materials for polymerization to obtain fluorine-containing polymers. For example, a fluorine-containing polymer obtained by polymerizing perfluoro(2-methylene-4-methyl-1,3-dioxolane), which is a fluorine-containing monomer with a ring structure, is useful for electronic components, optical materials, etc.

Specific fluorine-containing monomers are prone to polymerization during storage. Thus, in order to stably store such monomers, techniques for inhibiting polymerization have been reported (PTL 1 and PTL 2). PTL 1 discloses a monomer composition containing a fluorine-containing monomer, 2,6-di-t-butyl-p-cresol, and the like. PTL 2 discloses a method for stabilizing perfluoro(2-methylene-4-methyl-1,3-dioxolane) by incorporating a hydroxy group-containing fluoroaromatic compound of a specific structure into a composition containing perfluoro(2-methylene-4-methyl-1,3-dioxolane).

### CITATION LIST

### Patent Literature

PTL 1: WO2018/062193
PTL 2: WO2020/130122

### SUMMARY

The present disclosure encompasses, for example, the following aspects.

A composition comprising a fluorine-containing monomer (M) and fluoride ions,
wherein the fluoride ion content is 0.01 to 1000 ppm by mass based on the mass of the composition, and
the fluorine-containing monomer (M) is at least one monomer selected from the group consisting of
a compound represented by formula (M1):

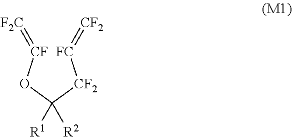

wherein R¹ and R² each independently represent a fluorine atom, a perfluoroalkyl group, or a perfluoroalkoxy group, a compound represented by formula (M2):

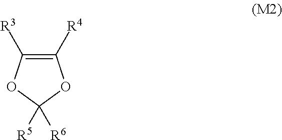

wherein R³, R⁴, R⁵, and R⁶ each independently represent a fluorine atom, a perfluoroalkyl group, or a perfluoroalkoxy group, and a compound represented by formula (M3):

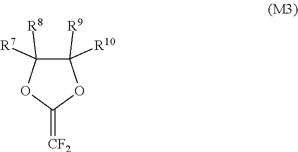

wherein R⁷, R⁸, R⁹, and R¹⁰ each independently represent a fluorine atom, a perfluoroalkyl group, or a perfluoroalkoxy group.

### Advantageous Effects

The present disclosure provides a method for reducing fluoride ions mixed in a specific fluorine-containing monomer (i.e., a method for purifying a fluorine-containing monomer). The use of this method enables the provision of a composition comprising a fluorine-containing monomer and fluoride ions wherein the amount of fluoride ions mixed in the fluorine-containing monomer is small, and enables the production of a fluorine-containing monomer having a further reduced fluoride ion content from a fluorine-containing monomer contaminated with fluoride ions.

### DESCRIPTION OF EMBODIMENTS

The above overview of the present disclosure is not intended to describe each of the disclosed embodiments or all of the implementations of the present disclosure.

The following description of the present disclosure more specifically provides examples of illustrative embodiments.

Guidance is provided through examples in several parts of the present disclosure, and these examples can be used in various combinations.

In each case, the group of examples can function as a non-exclusive and representative group.

All publications, patents, and patent applications cited herein are incorporated herein by reference in their entirety.

### Terms

Unless otherwise specified, the symbols and abbreviations in the present specification can be understood in the context of the present specification in the meanings commonly used in the technical field to which the present disclosure belongs.

In the present specification, the terms “comprise” and “contain” are used with the intention of including the phrases “consisting essentially of” and “consisting of.”

Unless otherwise specified, the steps, treatments, or operations described in the present specification may be performed at room temperature. In the present specification, room temperature can refer to a temperature within the range of 10 to 40° C.

In the present specification, the phrase “Cn-Cm” (n and m are each a number) indicates that the number of carbon atoms is n or more and m or less, as a person skilled in the art would generally understand.

In the present specification, the description of compounds can include all stereoisomers (enantiomers, diastereomers, geometric isomers, etc.) unless otherwise specified by a person skilled in the art.

In the present specification, unless otherwise specified, “alkyl” includes linear, branched, or cyclic alkyl groups. “Alkyl” may be linear or branched alkyl.

The number of carbon atoms in “alkyl” may be, for example, 1 to 12, 1 to 6, 1 to 5, 1 to 4, 1 to 3, 6, 5, 4, 3, 2, or 1.

Examples of alkyl include linear or branched alkyl groups, such as methyl, ethyl, propyl (e.g., n-propyl, isopropyl), butyl (e.g., n-butyl, isobutyl, sec-butyl, tert-butyl), pentyl (e.g., n-pentyl, tert-pentyl, neopentyl, isopentyl, sec-pentyl, 3-pentyl), hexyl, heptyl, octyl, nonyl, decyl, undecyl, and dodecyl; and cyclic alkyl groups, such as cyclopropyl, cyclobutyl, cyclopentyl, cyclohexyl, cycloheptyl, and cyclooctyl.

In the present specification, unless otherwise specified, “fluoroalkyl” includes linear, branched, or cyclic alkyl groups in which at least one hydrogen atom is replaced by a fluorine atom. “Fluoroalkyl” may be linear or branched alkyl.

The number of carbon atoms in “fluoroalkyl” may be, for example, 1 to 12, 1 to 6, 1 to 5, 1 to 4, 1 to 3, 6, 5, 4, 3, 2, or 1.

The number of fluorine atoms in “fluoroalkyl” may be 1 or more (e.g., 1 to 3, 1 to 5, 1 to 9, 1 to 11, or 1 to the maximum substitutable number).

“Fluoroalkyl” includes perfluoroalkyl. “Perfluoroalkyl” is alkyl in which all of the hydrogen atoms are replaced by fluorine atoms.

Examples of fluoroalkyl include linear or branched C1-C20 fluoroalkyl groups (for example, C1-C10, C1-C4, or C1-C3, preferably C1-C7, and more preferably C1-C6 fluoroalkyl (preferably perfluoroalkyl) groups), such as methyl having 1 to 3 fluorine atoms, ethyl having 1 to 5 fluorine atoms, propyl (e.g., n-propyl, isopropyl) having 1 to 7 fluorine atoms, butyl (e.g., n-butyl, isobutyl, sec-butyl, tert-butyl) having 1 to 9 fluorine atoms, pentyl (e.g., n-pentyl, tert-pentyl, neopentyl, isopentyl, sec-pentyl, 3-pentyl) having 1 to 11 fluorine atoms, hexyl having 1 to 13 fluorine atoms, heptyl having 1 to 15 fluorine atoms, octyl having 1 to 17 fluorine atoms, nonyl having 1 to 19 fluorine atoms, decyl having 1 to 21 fluorine atoms, undecyl having 1 to 23 fluorine atoms, dodecyl having 1 to 25 fluorine atoms, tridecyl having 1 to 27 fluorine atoms, tetradecyl having 1 to 29 fluorine atoms, pentadecyl having 1 to 31 fluorine atoms, hexadecyl having 1 to 33 fluorine atoms, heptadecyl having 1 to 35 fluorine atoms, octadecyl having 1 to 37 fluorine atoms, nonadecyl having 1 to 39 fluorine atoms, and icosyl having 1 to 41 fluorine atoms; and cyclic C3-C10 fluoroalkyl groups (for example, C3-C6, C4-C6, C3-C5, C5-C6, or C4-C8 fluoroalkyl (preferably perfluoroalkyl) groups), such as cyclofluoropropyl, cyclofluorobutyl, cyclofluoropentyl, cyclofluorohexyl, cyclofluoroheptyl, cyclofluorooctyl, and fluoroadamantyl.

Examples of perfluoroalkyl include trifluoromethyl (CF₃—), pentafluoroethyl (C₂F₅—), perfluoropropyl (e.g., CF₃CF₂CF₂— and (CF₃)₂CF—), perfluorobutyl (e.g., CF₃CF₂CF₂CF₂—, (CF₃)₂CFCF₂—, CF₃CF(CF₃) CF₂—, and (CF₃)₃C—), and perfluoropentyl (e.g., CF₃CF₂CF₂CF₂CF₂—, (CF₃)₂CFCF₂CF₂—, CF₃CF₂CF(CF₃) CF₂—, CF₃CF₂CF₂CF(CF₃)—, and CF₃C(CF₃)₂CF₂—).

Specific examples of fluoroalkyl include those listed above as examples, such as perfluoroalkyl, monofluoromethyl, difluoromethyl, 2,2,2-trifluoroethyl (CF₃CH₂—), tetrafluoropropyl (e.g., HCF₂CF₂CH₂—), hexafluoropropyl (e.g., (CF₃)₂CH—), and octafluoropentyl (e.g., HCF₂CF₂CF₂CF₂CH₂—).

In the present specification, unless otherwise indicated, “alkoxy” may be a group represented by RO— wherein R represents an alkyl group. “Alkoxy” includes linear, branched, or cyclic alkoxy groups. “Alkoxy” may be linear or branched alkoxy.

The number of carbon atoms in “alkoxy” may be, for example, 1 to 12, 1 to 6, 1 to 5, 1 to 4, 1 to 3, 6, 5, 4, 3, 2, or 1.

Examples of alkoxy include linear or branched alkoxy groups, such as methoxy, ethoxy, propoxy, isopropoxy, butoxy, isobutoxy, sec-butoxy, tert-butoxy, pentyloxy, isopentyloxy, neopentyloxy, hexyloxy, heptyloxy, octyloxy, nonyloxy, and decyloxy; and cyclic alkoxy, such as cyclopropyloxy, cyclobutyloxy, cyclopentyloxy, cyclohexyloxy, cycloheptyloxy, and cyclooctyloxy.

In the present specification, unless otherwise specified, “fluoroalkoxy” is alkoxy in which at least one hydrogen atom is replaced by a fluorine atom. “Fluoroalkoxy” may be linear or branched fluoroalkoxy.

The number of carbon atoms in “fluoroalkoxy” may be, for example, 1 to 12, 1 to 6, 1 to 5, 1 to 4, 1 to 3, 6, 5, 4, 3, 2, or 1.

The number of fluorine atoms in “fluoroalkoxy” may be 1 or more (e.g., 1 to 3, 1 to 5, 1 to 9, 1 to 11, or 1 to the maximum substitutable number).

“Fluoroalkoxy” includes perfluoroalkoxy. “Perfluoroalkoxy” is alkoxy in which all of the hydrogen atoms are replaced by fluorine atoms.

Examples of perfluoroalkoxy include trifluoromethyloxy (CF₃O—), pentafluoroethyloxy (C₂F₅O—), perfluoropropyloxy (e.g., CF₃CF₂CF₂O— and (CF₃)₂CFO—) perfluorobutyloxy (e.g., CF₃CF₂CF₂CF₂O—, (CF₃)₂CFCF₂O—, CF₃CF(CF₃)CF₂O—, and (CF₃)₃CO—), and perfluoropentyloxy (e.g., CF₃CF₂CF₂CF₂CF₂O—, (CF₃)₂CFCF₂CF₂O—, CF₃CF₂CF(CF₃)CF₂O—, CF₃CF₂CF₂CF(CF₃)O—, and CF₃C(CF₃)₂CF₂O—).

Specific examples of fluoroalkoxy include those listed as examples, such as perfluoroalkoxy, monofluoromethoxy, difluoromethoxy, 2,2,2-trifluoroethyloxy (CF₃CH₂O—), tetrafluoropropyloxy (e.g., HCF₂CF₂CH₂O—), hexafluoropropyloxy (e.g., (CF₃)₂CHO—), and octafluoropentyloxy (e.g., HCF₂CF₂CF₂CF₂CH₂O—).

### Composition

One embodiment of the present disclosure is a composition comprising a fluorine-containing monomer (M) and fluoride ions. The composition has a low fluoride ion content of, for example, 0.01 to 1000 ppm by mass based on the mass of the composition. Therefore, the composition of the present disclosure is useful as a source of a fluorine-containing monomer (M) in the production of a fluorine-containing polymer by polymerizing the fluorine-containing monomer (M). For example, the composition can be subjected to polymerization conditions either as is or after optionally removing or reducing fluoride ions contained in the composition to thereby produce a fluorine-containing polymer. Since the composition of the present disclosure has a low fluoride ion content, the corrosion of containers, piping, or the like that come into contact with the fluorine-containing monomer (M) can be prevented.

### Fluorine-Containing Monomer (M)

The fluorine-containing monomer (M) is at least one monomer selected from the group consisting of a compound represented by the following formula (M1):

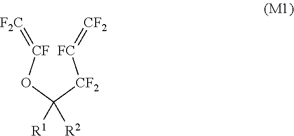

wherein R¹ and R² each independently represent a fluorine atom, a perfluoroalkyl group, or a perfluoroalkoxy group (which is also referred to as “monomer (M1)” in the present specification), a compound represented by formula (M2):

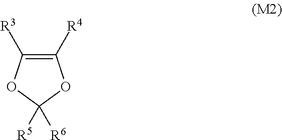

wherein R³, R⁴, R⁵, and R⁶ each independently represent a fluorine atom, a perfluoroalkyl group, or a perfluoroalkoxy group (which is also referred to as “monomer (M2)” in the present specification), and
a compound represented by formula (M3):

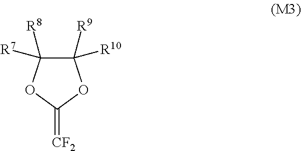

wherein R⁷, R⁸, R⁹, and R¹⁰ each independently represent a fluorine atom, a perfluoroalkyl group, or a perfluoroalkoxy group (which is also referred to as “monomer (M3)” in the present specification). The monomer (M) may be of one type or a combination of two or more types.

The production method for the fluorine-containing monomer (M) is known. In the present disclosure, the fluorine-containing monomer (M) may be produced by using a known production method. For example, the monomer (M3) can be produced according to the method described in, for example, JP2005-002014A, WO2020/166632A, or WO2020/230822A.

During storage, the fluorine-containing monomer (M) may undergo decomposition over time, forming fluoride ions, which may be mixed in the fluorine-containing monomer. In the present disclosure, the thus-obtained fluorine-containing monomer (M) is preferably used as a fluorine-containing monomer (M) contaminated with fluoride ions. The fluorine-containing monomer (M) contaminated with fluoride ions for use may also be a fluorine-containing monomer (M) containing fluoride ions that may be formed during the production of the fluorine-containing monomer (M). The fluorine-containing monomer (M) contaminated with fluoride ions may otherwise be obtained by adding fluoride ions to the fluorine-containing monomer (M).

### Monomer (M1)

R¹ and R² may each independently represent a fluorine atom, a C1-C5 perfluoroalkyl group, or a C1-C5 perfluoroalkoxy group.

R¹ and R² may each independently represent a fluorine atom, a C1-C4 perfluoroalkyl group, or a C1-C4 perfluoroalkoxy group.

R¹ and R² may each independently represent a fluorine atom, a C1-C3 perfluoroalkyl group, or a C1-C3 perfluoroalkoxy group.

R¹ and R² may each independently represent a fluorine atom, a C1-C2 perfluoroalkyl group, or a C1-C2 perfluoroalkoxy group.

R¹ and R² may each independently represent a fluorine atom, trifluoromethyl, pentafluoroethyl, or trifluoromethyloxy.

R¹ and R² may both be fluorine atoms.

A preferred monomer (M1) includes a compound represented by the following formula (M1-1) (which may also be referred to as “monomer (M1-1)” in the present specification).

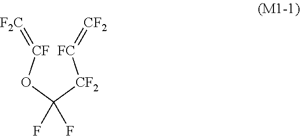

### Monomer (M2)

R³, R⁴, R⁵, and R⁶ may each independently represent a fluorine atom, a C1-C5 perfluoroalkyl group, or a C1-C5 perfluoroalkoxy group.

R³, R⁴, R⁵, and R⁶ may each independently represent a fluorine atom, a C1-C4 perfluoroalkyl group, or a C1-C4 perfluoroalkoxy group.

R³, R⁴, R⁵, and R⁶ may each independently represent a fluorine atom, a C1-C3 perfluoroalkyl group, or a C1-C3 perfluoroalkoxy group.

R³, R⁴, R⁵, and R⁶ may each independently represent a fluorine atom, trifluoromethyl, pentafluoroethyl, trifluoromethyloxy, or pentafluoroethyloxy.

R³, R⁴, R⁵, and R⁶ may each independently represent a fluorine atom, trifluoromethyl, pentafluoroethyl, or trifluoromethyloxy.

At least one group of R³, R⁴, R⁵, and R⁶ may represent a fluorine atom, and the remaining groups may, if plural, independently represent a C1-C5 perfluoroalkyl group or a C1-C2 perfluoroalkoxy group.

At least two groups of R³, R⁴, R⁵, and R⁶ may each represent a fluorine atom, and the remaining groups may, if plural, independently represent a C1-C5 perfluoroalkyl group or a C1-C2 perfluoroalkoxy group. In this case, it is preferable that R³ and R⁴ each represent a fluorine atom, and that R⁵ and R⁶ each independently represent a C1-C2 perfluoroalkyl group or a C1-C2 perfluoroalkoxy group.

At least three groups of R³, R⁴, R⁵, and R⁶ may each represent a fluorine atom, and the remaining group may represent a C1-C5 perfluoroalkyl group or a C1-C2 perfluoroalkoxy group. In this case, it is preferable that R³, R⁵, and R⁶ each represent a fluorine atom, and that R⁴ represents a C1-C5 perfluoroalkyl group or a C1-C2 perfluoroalkoxy group.

At least three groups of R³, R⁴, R⁵, and R⁶ may each represent a fluorine atom, and the remaining group may represent a C1-C2 perfluoroalkoxy group. In this case, it is preferable that R³, R⁵, and R⁶ each represent a fluorine atom, and that R⁴ represents a C1-C2 perfluoroalkoxy group.

R³, R⁴, R⁵, and R⁶ may all be fluorine atoms.

Preferable monomers (M2) include a compound represented by the following formula (M2-1) and a compound represented by the following formula (M2-2) (which are also referred to as “monomer (M2-1)” and “monomer (M2-2)” respectively in the present specification).

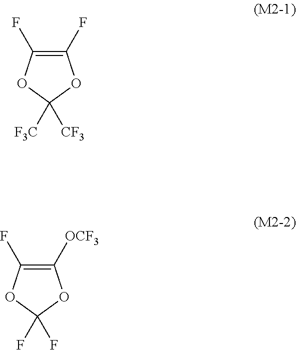 [SMILES: FC1=C(F)OC(C(F)(F)F)(C(F)(F)F)O1 ; FC1=C(OC(F)(F)F)OC(F)(F)O1]

### Monomer (M3)

R⁷, R⁸, R⁹, and R¹⁰ may each independently represent a fluorine atom, a C1-C5 perfluoroalkyl group, or a C1-C5 perfluoroalkoxy group.

R⁷, R⁸, R⁹, and R¹⁰ may each independently represent a fluorine atom, a C1-C4 perfluoroalkyl group, or a C1-C4 perfluoroalkoxy group.

R⁷, R⁸, R⁹, and R¹⁰ may each independently represent a fluorine atom, a C1-C3 perfluoroalkyl group, or a C1-C3 perfluoroalkoxy group.

R⁷, R⁸, R⁹, and R¹⁰ may each independently represent a fluorine atom, trifluoromethyl, pentafluoroethyl, trifluoromethyloxy, or pentafluoroethyloxy.

R⁷, R⁸, R⁹, and R¹⁰ may each independently represent fluorine atom, trifluoromethyl, pentafluoroethyl, or trifluoromethyloxy.

At least one group of R⁷, R⁸, R⁹, and R¹⁰ may represent a fluorine atom, and the remaining groups may, if plural, independently represent a C1-C2 perfluoroalkyl group or a C1-C2 perfluoroalkoxy group.

At least two groups of R⁷, R⁸, R⁹, and R¹⁰ may each represent a fluorine atom, and the remaining groups may, if plural, independently represent a C1-C2 perfluoroalkyl group or a C1-C2 perfluoroalkoxy group.

At least three groups of R⁷, R⁸, R⁹, and R¹⁰ may each represent a fluorine atom, and the remaining group may represent a C1-C2 perfluoroalkyl group or a C1-C2 perfluoroalkoxy group.

At least three groups of R⁷, R⁸, R⁹, and R¹⁰ may each represent a fluorine atom, and the remaining group may represent a C1-C2 perfluoroalkyl group.

R⁷, R⁸, R⁹, and R¹⁰ may all be fluorine atoms.

The monomer (M3) is preferably a compound represented by the following formula (M3-1) (perfluoro(2-methylene-4-methyl-1,3-dioxolane); also referred to as “monomer (M3-1)” in the present specification) or a compound represented by the following formula (M3-2) (perfluoro(2-methylene-1,3-dioxolane); also referred to as “monomer (M3-2)” in the present specification).

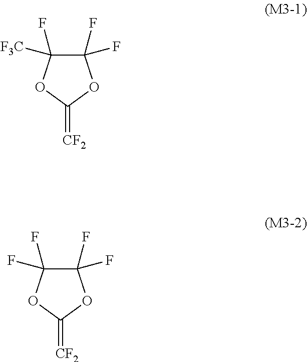

In the composition of the present disclosure, the content of the fluorine-containing monomer (M) may be, for example, 89.9 to 99.99 mass %, preferably 91 to 99.99 mass %, and more preferably 92 to 99.99 mass %, based on the mass of the composition.

### Fluoride Ion

The composition of the present disclosure comprises fluoride ions, in addition to the fluorine-containing monomer (M). Generally, the fluorine-containing monomer (M) undergoes decomposition during storage and forms fluoride ions. The presence of fluoride ions causes disadvantages, such as reducing the thermal stability of a polymer obtained by polymerizing the fluorine-containing monomer (M), inducing corrosion in production equipment, increasing the cost of wastewater treatment, and requiring additional steps for removal or reduction of fluoride ions. However, the fluoride ion content is low in the composition of the present disclosure. In the composition of the present disclosure, the fluoride ion content can be, for example, 0.01 to 1000 ppm by mass, preferably 0.01 to 800 ppm by mass, more preferably 0.01 to 600 ppm by mass, and most preferably 50 to 600 ppm by mass, based on the mass of the composition.

### Method for Specifying Fluoride Ion Content

The fluoride ion content of the fluorine-containing monomer (M) can be specified as follows.

Pure water (5 mL) is added to 1 g of the monomer (M), and the mixture is stirred at room temperature. The resulting aqueous phase is separated, and 4 mL of total ionic strength adjustment buffer is added to 4 mL of the aqueous phase. The fluoride ion concentration in the produced solution is measured using an ion meter. Taking the dilution rate into account, a value ten times the obtained measurement value is determined as the fluoride ion (HF) concentration.

### Compound Represented by Formula (C)

The composition of the present disclosure may comprise a compound represented by formula (C):

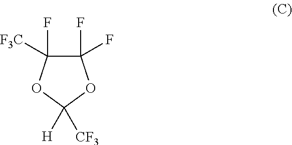 [SMILES: FC(F)(F)C1OC(F)(F)C(F)(C(F)(F)F)O1]

(which may also be referred to as “compound (C)” in the present specification).

In the composition of the present disclosure, the content of compound (C) may be, for example, 0.01 to 10 mass %, preferably 0.01 to 8 mass %, and more preferably 0.01 to 6 mass %, based on the mass of the composition.

In addition to the fluorine-containing monomer (M) and fluoride ions, the composition of the present disclosure may further comprise additional components other than the compound (C). The additional components may include impurities and the like that are mixed in during the production process of the fluorine-containing monomer (M). The content of the additional components may be, for example, 0.001 to 8 mass %, 0.001 to 6 mass %, or 0.001 to 5 mass %, based on the mass of the composition.

The composition of the present disclosure can be obtained, for example, by applying the purification method described below to the fluorine-containing monomer (M) contaminated with fluoride ions. The composition of the present disclosure may also be produced by adding fluoride ions to a fluorine-containing monomer (M) that is not contaminated with fluoride ions.

### Method for Purifying Fluorine-Containing Monomer (M)

Methods for purifying a fluorine-containing monomer (M) contaminated with fluoride ions had not been studied. The purification method of the present disclosure is a method for purifying a fluorine-containing monomer (M) contaminated with fluoride ions, and comprises subjecting the fluorine-containing monomer (M) contaminated with fluoride ions to at least one treatment selected from the group consisting of activated carbon adsorption and washing to reduce fluoride ions. This step of the purification method of the present disclosure can effectively reduce fluoride ions in the fluorine-containing monomer (M).

In the step of subjecting the fluorine-containing monomer (M) contaminated with fluoride ions to activated carbon adsorption treatment to reduce fluoride ions (activated carbon adsorption step), for example, the fluorine-containing monomer (M) contaminated with fluoride ions is brought into contact with activated carbon to allow the fluoride ions to be adsorbed onto the activated carbon. Then, by separating the activated carbon, a fluorine-containing monomer (M) having a reduced fluoride ion content can be obtained.

The fluoride ion content of the fluorine-containing monomer (M) contaminated with fluoride ions may be, for example, 0.01 to 1000 ppm by mass, 0.01 to 800 ppm by mass, or 0.01 to 600 ppm by mass.

The activated carbon adsorption treatment is not limited as long as the fluorine-containing monomer (M) contaminated with fluoride ions is brought into contact with activated carbon. Activated carbon may be added to the fluorine-containing monomer (M) contaminated with fluoride ions. Alternatively, the fluorine-containing monomer (M) contaminated with fluoride ions may be passed through a column or the like packed with activated carbon. After the activated carbon adsorption treatment, the activated carbon can be removed from the fluorine-containing monomer (M) by a known solid-liquid separation method.

The amount of activated carbon used in the activated carbon adsorption step may be, for example, 1 g or more, or 1 to 20 g, and preferably 1 to 10 g, per 100 g of the fluorine-containing monomer (M) contaminated with fluoride ions.

The temperature of the activated carbon adsorption treatment is, for example, −40 to 25° C., and preferably −20 to 25° C.

The fluorine-containing monomer (M) purified according to the purification method of the present disclosure may contain the compound (C). The content of the compound represented by formula (C) in the purified fluorine-containing monomer (M) may be 100 to 100000 ppm by mass.

In the purification method of the present disclosure, a step of distilling the fluorine-containing monomer (M) contaminated with fluoride ions (distillation step) may be provided before the activated carbon adsorption step. Distilling the fluorine-containing monomer (M) contaminated with fluoride ions is advantageous because high-boiling components, such as oligomers, can be removed from the fluorine-containing monomer (M).

The distillation treatment can be performed, for example, by applying a known distillation method to the fluorine-containing monomer (M) contaminated with fluoride ions. The distillation temperature is, for example, 30° C. to 60° C., and preferably 30° C. to 50° C. The distillation pressure is, for example, 50 to 500 hPa, and preferably 200 to 500 hPa. The evaporated component produced during distillation is liquefied by cooling (for example, at −78° C. to −20° C.), and the obtained liquid is subjected to the activated carbon adsorption step.

In the step of subjecting the fluorine-containing monomer (M) contaminated with fluoride ions to washing treatment to reduce fluoride ions (washing step), for example, the fluorine-containing monomer (M) contaminated with fluoride ions may be brought into contact with a washing liquid to extract the fluoride ions into the aqueous phase, the aqueous phase is then separated and removed to obtain a non-aqueous phase, whereby a fluorine-containing monomer (M) having a reduced amount of fluoride ions mixed therein can be obtained. The washing step may be performed once or multiple times, and is preferably performed 1 to 3 times.

The washing liquid used in the washing step may be water, an aqueous KOH solution, an aqueous K₂CO₃ solution, an aqueous KHCO₃ solution, an aqueous NaOH solution, an aqueous NaHCO₃ solution, an aqueous Na₂CO₃ solution, an aqueous CsOH solution, an aqueous Cs₂CO₃ solution, an aqueous Ca(OH)₂ solution, an aqueous Ba(OH)₂ solution, or the like. The washing liquid is preferably pure water, an aqueous KOH solution, an aqueous K₂CO₃ solution, or an aqueous KHCO₃ solution, and more preferably pure water.

The amount of the washing liquid used in the washing step may be, for example, 20 g or more, or 20 to 500 g, and preferably 50 to 200 g, per 100 g of the fluorine-containing monomer (M) contaminated with fluoride ions.

The temperature of the washing treatment is, for example, 5 to 40° C., and preferably 10 to 30° C.

In the washing step, a drying agent (such as molecular sieves, silica gel, calcium chloride, or calcium oxide) may be brought into contact with the non-aqueous phase obtained during the washing treatment to remove moisture remaining in the non-aqueous phase. The drying agent can be removed from the non-aqueous phase by a known solid-liquid separation method. The amount of the drying agent used may be appropriately selected. For example, the amount may be 1 to 20 g per 100 g of the non-aqueous phase.

### Method for Producing Fluorine-Containing Monomer (M) Having Further Reduced Fluoride Ion Content

The production method of the present disclosure is a method for producing a fluorine-containing monomer (M) having a further reduced fluoride ion content (for example, a fluorine-containing monomer (M) having a fluoride ion content of 0.01 to 1000 ppm by mass) from a fluorine-containing monomer (M) contaminated with fluoride ions. The method comprises subjecting the fluorine-containing monomer (M) contaminated with fluoride ions to at least one treatment selected from the group consisting of activated carbon adsorption and washing to reduce fluoride ions (fluoride ion reduction step).

The fluoride ion reduction step can be performed, for example, by applying the method for purifying a fluorine-containing monomer (M) described above to the fluorine-containing monomer (M) contaminated with fluoride ions. Thus, the description above regarding the method for purifying a fluorine-containing monomer (M) may be applied to the fluoride ion reduction step to the possible extent.

The fluoride ion content of the fluorine-containing monomer (M) produced according to the production method of the present disclosure is, for example, 0.01 to 1000 ppm by mass, preferably 0.01 to 800 ppm by mass, and more preferably 0.01 to 600 ppm by mass.

The embodiments are described above; however, it will be understood that various changes in forms and details can be made without departing from the spirit and scope of the claims. The present disclosure encompasses, for example, the following aspects.

### Item 1.

A composition comprising a fluorine-containing monomer (M) and fluoride ions,
wherein the fluoride ion content is 0.01 to 1000 ppm by mass based on the mass of the composition, and
the fluorine-containing monomer (M) is at least one monomer selected from the group consisting of
a compound represented by formula (M1):

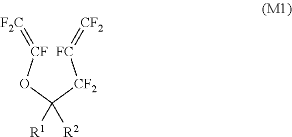

wherein R¹ and R² each independently represent a fluorine atom, a perfluoroalkyl group, or a perfluoroalkoxy group,
a compound represented by formula (M2):

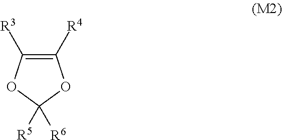

wherein R³, R⁴, R⁵, and R⁶ each independently represent a fluorine atom, a perfluoroalkyl group, or a perfluoroalkoxy group, and a compound represented by formula (M3):

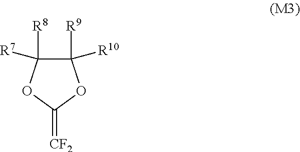

wherein R⁷, R⁸, R⁹, and R¹⁰ each independently represent a fluorine atom, a perfluoroalkyl group, or a perfluoroalkoxy group.

### Item 2.

The composition according to Item 1, wherein the fluorine-containing monomer (M) is at least one compound selected from the group consisting of a compound represented by the following formula (M1-1), a compound represented by the following formula (M2-1), a compound represented by the following formula (M2-2), a compound represented by the following formula (M3-1), and a compound represented by the following formula (M3-2):

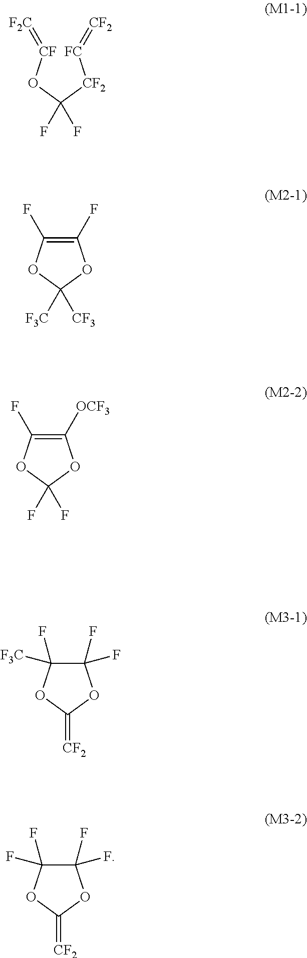

### Item 3.

The composition according to Item 1 or 2, wherein the fluorine-containing monomer (M) is a compound represented by the following formula (M3-1):

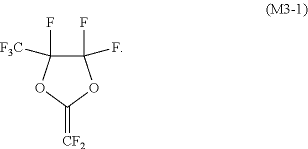

### Item 4.

The composition according to Item 3, further comprising a compound represented by the following formula (C):

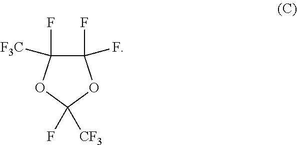 [SMILES: FC(F)(F)C1(F)OC(F)(F)C(F)(C(F)(F)F)O1]

### Item 5.

The composition according to Item 4, wherein the content of the compound represented by formula (C) is 0.01 to 10 mass % based on the mass of the composition.

### Item 6.

The composition according to any one of Items 1 to 5, wherein the fluoride ion content is 50 to 600 ppm by mass based on the mass of the composition.

### Item 7.

The composition according to Item 1, wherein the fluorine-containing monomer (M) is a compound represented by the following formula (M3-1):

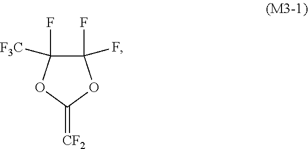

and
the fluoride ion content is 50 to 600 ppm by mass based on the mass of the composition.

### Item 8.

A method for producing a fluorine-containing monomer (M) having a further reduced fluoride ion content from a fluorine-containing monomer (M) contaminated with fluoride ions,
the method comprising:
subjecting the fluorine-containing monomer (M) contaminated with fluoride ions to at least one treatment selected from the group consisting of activated carbon adsorption and washing to reduce fluoride ions;
   wherein the fluoride ion content of the fluorine-containing monomer (M) produced is 0.01 to 1000 ppm by mass, and the fluorine-containing monomer (M) is at least one monomer selected from the group consisting of
   a compound represented by formula (M1):

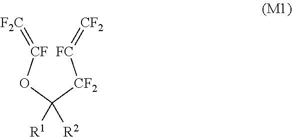

wherein R¹ and R² each independently represent a fluorine atom, a perfluoroalkyl group, or a perfluoroalkoxy group, a compound represented formula (M2):

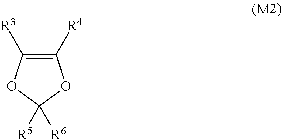

wherein R³, R⁴, R⁵, and R⁶ each independently represent a fluorine atom, a perfluoroalkyl group, or a perfluoroalkoxy group, and a compound represented by formula (M3):

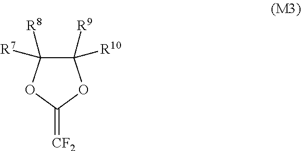

wherein R⁷, R⁸, R⁹, and R¹⁰ each independently represent a fluorine atom, a perfluoroalkyl group, or a perfluoroalkoxy group.

### Item 9.

The production method according to Item 8, wherein the treatment of the fluorine-containing monomer (M) contaminated with fluoride ions is activated carbon adsorption.

### Item 10.

The production method according to Item 9, wherein the activated carbon adsorption treatment comprises distilling the fluorine-containing monomer (M) contaminated with fluoride ions and subjecting the distilled fluorine-containing monomer (M) to activated carbon adsorption treatment.

### Item 11.

The production method according to Item 8, wherein the washing is performed with water or an alkaline aqueous solution.

### Item 12.

The production method according to any one of Items 8 to 11, wherein the fluorine-containing monomer (M) is at least one compound selected from the group consisting of a compound represented by the following formula (M1-1), a compound represented by the following formula (M2-1), a compound represented by the following formula (M2-2), a compound represented by the following formula (M3-1), and a compound represented by the following formula (M3-2):

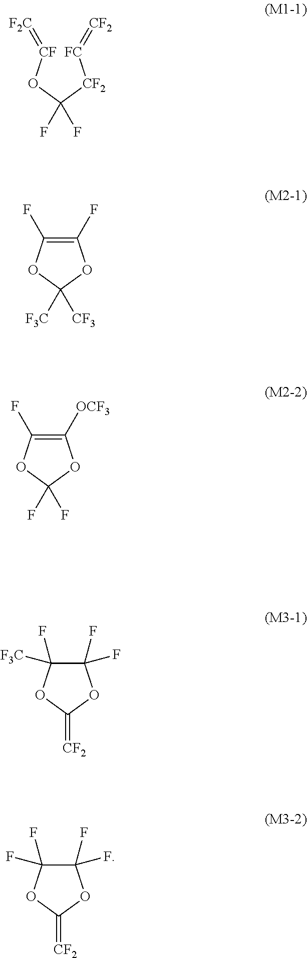

### Item 13.

The production method according to any one of Items 8 to 11, wherein the fluorine-containing monomer (M) is a compound represented by the following formula (M3-1):

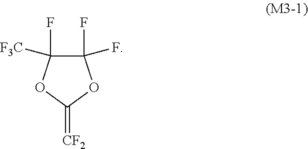

### Item 14.

The production method according to any one of Items 8 to 13, wherein the fluoride ion content of the fluorine-containing monomer (M) produced is 50 to 600 ppm by mass.

### Item 15.

The production method according to Item 8, wherein the fluorine-containing monomer (M) is a compound represented by the following formula (M3-1):

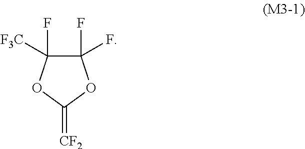

the treatment of the fluorine-containing monomer (M) contaminated with fluoride ions comprises (1) subjecting the fluorine-containing monomer (M) contaminated with fluoride ions to activated carbon adsorption treatment, (2) distilling the fluorine-containing monomer (M) contaminated with fluoride ions and subjecting the distilled fluorine-containing monomer (M) to activated carbon adsorption treatment, or (3) washing the fluorine-containing monomer (M) contaminated with fluoride ions with water or an alkaline aqueous solution, and the fluoride ion content of the fluorine-containing monomer (M) produced is 50 to 600 ppm by mass.

### Item 16.

A method for purifying a fluorine-containing monomer (M) contaminated with fluoride ions,
the method comprising:
subjecting the fluorine-containing monomer (M) contaminated with fluoride ions to at least one treatment selected from the group consisting of activated carbon adsorption and washing to reduce fluoride ions;
   wherein the fluorine-containing monomer (M) is at least one monomer selected from the group consisting of
   a compound represented by formula (M1):

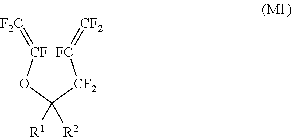

wherein R¹ and R² each independently represent a fluorine atom, a perfluoroalkyl group, or a perfluoroalkoxy group, a compound represented by formula (M2):

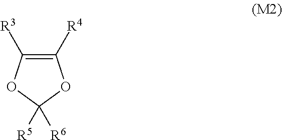

wherein R³, R⁴, R⁵, and R⁶ each independently represent a fluorine atom, a perfluoroalkyl group, or a perfluoroalkoxy group, and a compound represented by formula (M3):

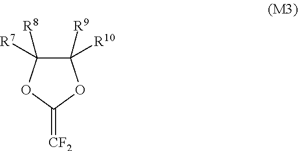

wherein R⁷, R⁸, R⁹, and R¹⁰ each independently represent a fluorine atom, a perfluoroalkyl group, or a perfluoroalkoxy group.

### Item 17.

The purification method according to Item 16, wherein the fluoride ion content of a fluorine-containing monomer (M) purified is 0.01 to 1000 ppm by mass.

### Item 18.

The purification method according to Item 16, wherein the fluoride ion content of a fluorine-containing monomer (M) purified is 50 to 600 ppm by mass.

### Item 19.

The purification method according to Item 16, wherein the treatment of the fluorine-containing monomer (M) contaminated with fluoride ions is activated carbon adsorption.

### Item 20.

The purification method according to Item 19, wherein the activated carbon adsorption treatment comprises distilling the fluorine-containing monomer (M) contaminated with fluoride ions and subjecting the distilled fluorine-containing monomer (M) to activated carbon adsorption treatment.

### Item 21.

The purification method according to Item 16, wherein the washing is performed with water or an alkaline aqueous solution.

### Item 22.

The purification method according to any one of Items 16 to 21, wherein the fluorine-containing monomer (M) is at least one compound selected from the group consisting of a compound represented by the following formula (M1-1), a compound represented by the following formula (M2-1), a compound represented by the following formula (M2-2), a compound represented by the following formula (M3-1), and a compound represented by the following formula (M3-2):

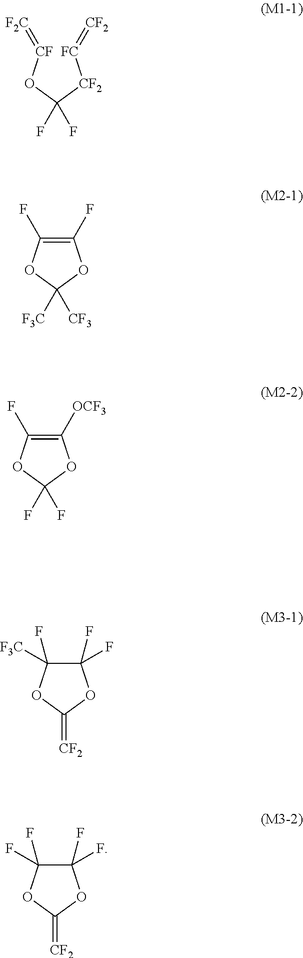

### Item 23.

The purification method according to any one of Items 16 to 21, wherein the fluorine-containing monomer (M) is a compound represented by the following formula (M3-1):

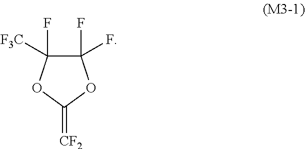

### Item 24.

The purification method according to Item 23, wherein the fluorine-containing monomer (M) contaminated with fluoride ions is a fluorine-containing monomer contaminated with fluoride ions and a compound represented by the following formula (C):

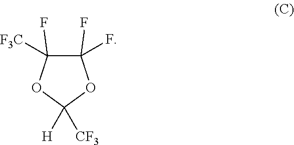 [SMILES: FC(F)(F)C1OC(F)(F)C(F)(C(F)(F)F)O1]

### Item 25.

The purification method according to Item 24, wherein the content of the compound represented by formula (C) in the fluorine-containing monomer (M) purified is 100 to 100000 ppm by mass.

### Item 26.

The purification method according to Item 16, wherein the fluorine-containing monomer (M) is a compound represented by the following formula (M3-1):

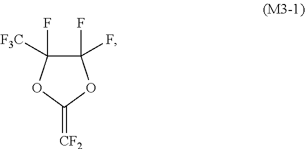

the fluorine-containing monomer (M) contaminated with fluoride ions is a fluorine-containing monomer contaminated with fluoride ions and a compound represented by the following formula (C):

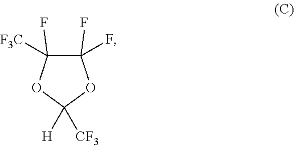 [SMILES: FC(F)(F)C1OC(F)(F)C(F)(C(F)(F)F)O1]

the treatment of the fluorine-containing monomer (M) contaminated with fluoride ions comprises (1) subjecting the fluorine-containing monomer (M) contaminated with fluoride ions to activated carbon adsorption treatment, (2) distilling the fluorine-containing monomer (M) contaminated with fluoride ions and subjecting the distilled fluorine-containing monomer (M) to activated carbon adsorption treatment, or (3) washing the fluorine-containing monomer (M) contaminated with fluoride ions with water or an alkaline aqueous solution, the fluoride ion content of a fluorine-containing monomer (M) purified is 50 to 600 ppm by mass, and the content of the compound represented by formula (C) in the fluorine-containing monomer (M) purified is 100 to 100000 ppm by mass.

### EXAMPLES

The following describes an embodiment of the present disclosure in more detail with reference to Examples and the like. However, the present disclosure is not limited to these Examples.

The fluorine-containing monomer etc. used in the following Examples are as follows.

### Fluorine-Containing Monomer

Perfluoro(2-methylene-4-methyl-1,3-dioxolane) (Monomer (M3-1))

### Adsorbent; Activated Carbon

Shirasagi A (Osaka Gas Chemical Co., Ltd.)

Adsorbent; molecular sieves

Molecular Sieves 4A 1/16 (FUJIFILM Wako Pure Chemical Corporation)

### Adsorbent; Silica Gel

Wakogel (trademark) C-300 (FUJIFILM Wako Pure Chemical Corporation)

### Adsorbent; Alumina

Activated alumina (FUJIFILM Wako Pure Chemical Corporation)

### Method for Measuring the Fluoride Ion Content of Fluorine-Containing Monomer

The fluoride ion content of a fluorine-containing monomer was measured as follows.

Pure water (5 mL) was added to 1 g of a fluorine-containing monomer, and the mixture was stirred at room temperature. The produced aqueous phase was separated, and 4 mL of total ionic strength adjustment buffer was added to 4 mL of the aqueous phase. The fluoride ion concentration in the produced solution was measured using an ion meter. Taking into account the dilution rate, a value ten times the obtained measurement value was determined as the fluoride ion (HF) content.

### Distillation Treatment

The distillation treatment was performed as follows.

A fluorine-containing monomer was placed in a glass flask, distilled at a temperature of 40° C. and a pressure of 400 hPa, and trapped in acetone cooled to −78° C. with dry ice, thereby obtaining a fluorine-containing monomer.

### Activated Carbon Adsorption Treatment

Activated carbon (0.5 g) was added to 10 g of a fluorine-containing monomer, and the mixture was shaken at 0° C. for 10 minutes. The fluorine-containing monomer was filtered through a syringe filter to separate and remove the activated carbon, thereby obtaining a purified fluorine-containing monomer.

### Washing Treatment

Pure water or a 5% (w/v) aqueous KOH solution was used as a washing liquid. The washing liquid (10 g) was added to 10 g of a fluorine-containing monomer, and the mixture was stirred. The produced aqueous phase was removed to obtain a non-aqueous phase. An additional 10 g of the washing liquid was added to the non-aqueous phase to obtain a non-aqueous phase in a similar manner. An additional 10 g of the washing liquid was added to the non-aqueous phase to obtain a non-aqueous phase in a similar manner. The stirring (washing) process after the addition of the washing liquid was performed three times. Molecular sieves (0.5 g) were then added to the obtained non-aqueous phase and stirred to reduce the remaining components of the washing liquid. The obtained non-aqueous phase was filtered through a syringe filter to separate and remove the molecular sieves, thereby obtaining a purified fluorine-containing monomer.

### Production Example 1 (Preparation of Monomer (M3-1))

A monomer (M3-1) was produced according to a known method. The fluoride ion content of the obtained monomer (M3-1) was 3000 ppm by mass.

### Example 1 (Activated Carbon Adsorption Treatment)

The monomer (M3-1) obtained in Production Example 1 was subjected to activated carbon adsorption treatment. The fluoride ion content of the obtained monomer (M3-1) was 500 ppm by mass. The fluoride ion content was reduced from 3000 ppm by mass to 500 ppm by mass.

### Example 2 (Distillation and Activated Carbon Adsorption Treatment)

The monomer (M3-1) obtained in Production Example 1 was subjected to distillation treatment and then to activated carbon adsorption treatment for purification. The fluoride ion content of the obtained monomer (M3-1) was 80 ppm by mass. The fluoride ion content was reduced from 3000 ppm by mass to 80 ppm by mass.

### Example 3 (Washing Treatment with Water)

The monomer (M3-1) obtained in Production Example 1 was subjected to washing treatment with pure water. The fluoride ion content of the obtained monomer (M3-1) was 340 ppm by mass. The fluoride ion content was reduced from 3000 ppm by mass to 340 ppm by mass.

### Example 4 (Washing Treatment with 5% Aqueous KOH Solution)

The monomer (M3-1) obtained in Production Example 1 was subjected to washing treatment with a 5% aqueous KOH solution. The fluoride ion content of the obtained monomer (M3-1) was 290 ppm by mass. The fluoride ion content was reduced from 3000 ppm by mass to 290 ppm by mass.

### Comparative Examples 1 to 3 (Distillation and Adsorption Treatment)

The monomer (M3-1) obtained in Production Example 1 was purified in the same manner as in Example 2, except that the adsorbent was changed from activated carbon to molecular sieves, silica gel, or alumina. The fluoride ion content of the obtained monomer (M3-1) was 2780 ppm by mass, 1340 ppm by mass, and 1900 ppm by mass, respectively.

The results of Examples 1 to 4 and Comparative Examples 1 to 3 are shown in Table 1. In Examples 1 to 4, the fluoride ion content of the fluorine-containing monomer was significantly reduced.

TABLE 1

| | | | Fluoride ion content | |
|---|---|---|---|---|
| | | | (ppm by mass) | |
| | | | before | after |
| | Treatment | | treatment | treatment |
| Example 1 | Adsorption | | 3000 | 500 |
| | (activated carbon) | | | |
| Example 2 | Distillation | Adsorption | 3000 | 80 |
| | | (activated carbon) | | |
| Example 3 | Washing with water | | 3000 | 340 |
| Example 4 | Washing with KOH | | 3000 | 290 |
| Comparative | Distillation | Adsorption | 3000 | 2780 |
| Example 1 | | (molecular sieves) | | |
| Comparative | Distillation | Adsorption | 3000 | 1340 |
| Example 2 | | (silica gel) | | |
| Comparative | Distillation | Adsorption | 3000 | 1900 |
| Example 3 | | (alumina) | | |

1. A composition comprising a fluorine-containing monomer (M) and fluoride ions,
wherein the fluoride ion content is 0.01 to 1000 ppm by mass based on the mass of the composition, and
the fluorine-containing monomer (M) is at least one monomer selected from the group consisting of
a compound represented by formula (M1): 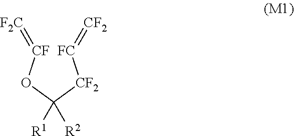
wherein R¹ and R² each independently represent a fluorine atom, a perfluoroalkyl group, or a perfluoroalkoxy group,
a compound represented by formula (M2): 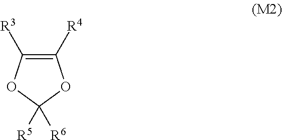
wherein R³, R⁴, R⁵, and R⁶ each independently represent a fluorine atom, a perfluoroalkyl group, or a perfluoroalkoxy group, and
a compound represented by formula (M3): 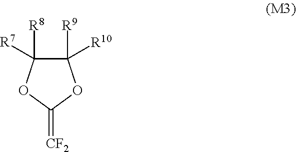
wherein R⁷, R⁸, R⁹, and R¹⁰ each independently represent a fluorine atom, a perfluoroalkyl group, or a perfluoroalkoxy group.

2. The composition according to claim 1, wherein the fluorine-containing monomer (M) is at least one compound selected from the group consisting of a compound represented by the following formula (M1-1), a compound represented by the following formula (M2-1), a compound represented by the following formula (M2-2), a compound represented by the following formula (M3-1), and a compound represented by the following formula (M3-2):

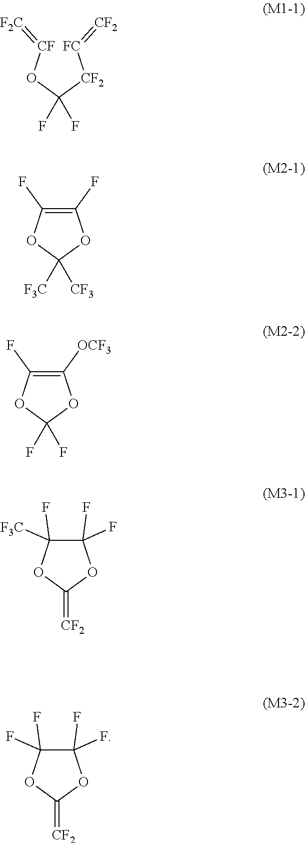

3. The composition according to claim 1, wherein the fluorine-containing monomer (M) is a compound represented by the following formula (M3-1):

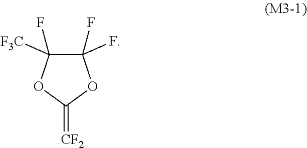

4. The composition according to claim 3, further comprising a compound represented by the following formula (C):

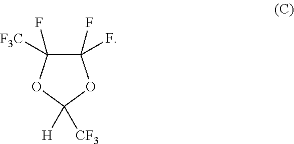 [SMILES: FC(F)(F)C1OC(F)(F)C(F)(C(F)(F)F)O1]

5. The composition according to claim 4, wherein the content of the compound represented by formula (C) is 0.01 to 10 mass % based on the mass of the composition.

6. The composition according to claim 1, wherein the fluoride ion content is 50 to 600 ppm by mass based on the mass of the composition.

7. The composition according to claim 1, wherein the fluorine-containing monomer (M) is a compound represented by the following formula (M3-1): 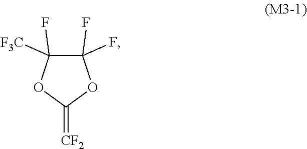
and the fluoride ion content is 50 to 600 ppm by mass based on the mass of the composition.

8. A method for producing a fluorine-containing monomer (M) having a further reduced fluoride ion content from a fluorine-containing monomer (M) contaminated with fluoride ions,
the method comprising:
subjecting the fluorine-containing monomer (M) contaminated with fluoride ions to at least one treatment selected from the group consisting of activated carbon adsorption and washing to reduce fluoride ions;
wherein the fluoride ion content of the fluorine-containing monomer (M) produced is 0.01 to 1000 ppm by mass, and
the fluorine-containing monomer (M) is at least one monomer selected from the group consisting of
a compound represented by formula (M1): 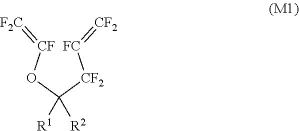
wherein R¹ and R² each independently represent a fluorine atom, a perfluoroalkyl group, or a perfluoroalkoxy group,
a compound represented formula (M2): 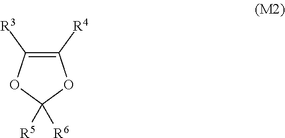
wherein R³, R⁴, R⁵, and R⁶ each independently represent a fluorine atom, a perfluoroalkyl group, or a perfluoroalkoxy group, and
a compound represented by formula (M3): 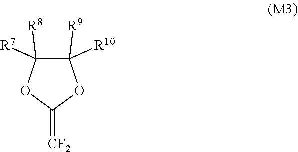
wherein R⁷, R¹, R⁹, and R¹⁰ each independently represent a fluorine atom, a perfluoroalkyl group, or a perfluoroalkoxy group.

9. The production method according to claim 8, wherein the treatment of the fluorine-containing monomer (M) contaminated with fluoride ions is activated carbon adsorption.

10. The production method according to claim 9, wherein the activated carbon adsorption treatment comprises distilling the fluorine-containing monomer (M) contaminated with fluoride ions and subjecting the distilled fluorine-containing monomer (M) to activated carbon adsorption treatment.

11. The production method according to claim 8, wherein the washing is performed with water or an alkaline aqueous solution.

12. The production method according to claim 8, wherein the fluorine-containing monomer (M) is at least one compound selected from the group consisting of a compound represented by the following formula (M1-1), a compound represented by the following formula (M2-1), a compound represented by the following formula (M2-2), a compound represented by the following formula (M3-1), and a compound represented by the following formula (M3-2):

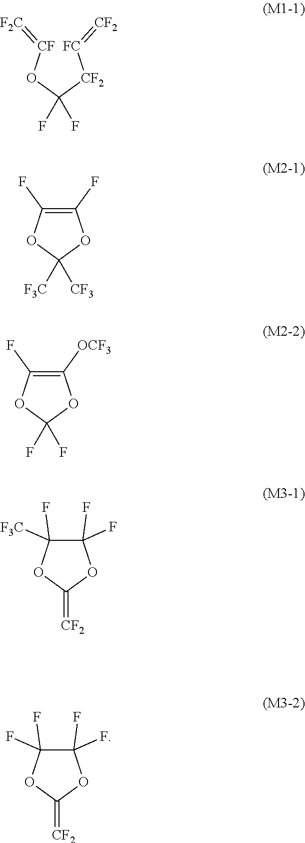

13. The production method according to claim 8, wherein the fluorine-containing monomer (M) is a compound represented by the following formula (M3-1):

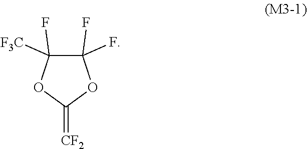

14. The production method according to claim 8, wherein the fluoride ion content of the fluorine-containing monomer (M) produced is 50 to 600 ppm by mass.

15. The production method according to claim 8,
wherein the fluorine-containing monomer (M) is a compound represented by the following formula (M3-1): 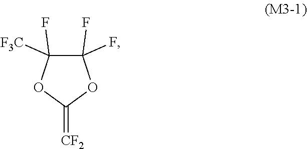
the treatment of the fluorine-containing monomer (M) contaminated with fluoride ions comprises (1) subjecting the fluorine-containing monomer (M) contaminated with fluoride ions to activated carbon adsorption treatment, (2) distilling the fluorine-containing monomer (M) contaminated with fluoride ions and subjecting the distilled fluorine-containing monomer (M) to activated carbon adsorption treatment, or (3) washing the fluorine-containing monomer (M) contaminated with fluoride ions with water or an alkaline aqueous solution, and
the fluoride ion content of the fluorine-containing monomer (M) produced is 50 to 600 ppm by mass.

16. A method for purifying a fluorine-containing monomer (M) contaminated with fluoride ions,
the method comprising:
subjecting the fluorine-containing monomer (M) contaminated with fluoride ions to at least one treatment selected from the group consisting of activated carbon adsorption and washing to reduce fluoride ions;
wherein the fluorine-containing monomer (M) is at least one monomer selected from the group consisting of
a compound represented by formula (M1): 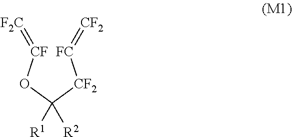
wherein R¹ and R² each independently represent a fluorine atom, a perfluoroalkyl group, or a perfluoroalkoxy group,
a compound represented by formula (M2): 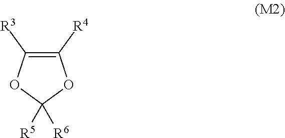
wherein R³, R⁴, R⁵, and R⁶ each independently represent a fluorine atom, a perfluoroalkyl group, or a perfluoroalkoxy group, and
a compound represented by formula (M3): 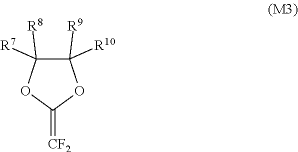
wherein R⁷, R¹, R⁹, and R¹⁰ each independently represent a fluorine atom, a perfluoroalkyl group, or a perfluoroalkoxy group.

17. The purification method according to claim 16, wherein the fluoride ion content of a fluorine-containing monomer (M) purified is 0.01 to 1000 ppm by mass.

18. The purification method according to claim 16, wherein the fluoride ion content of a fluorine-containing monomer (M) purified is 50 to 600 ppm by mass.

19. The purification method according to claim 16, wherein the treatment of the fluorine-containing monomer (M) contaminated with fluoride ions is activated carbon adsorption.

20. The purification method according to claim 19, wherein the activated carbon adsorption treatment comprises distilling the fluorine-containing monomer (M) contaminated with fluoride ions and subjecting the distilled fluorine-containing monomer (M) to activated carbon adsorption treatment.

21. The purification method according to claim 16, wherein the washing is performed with water or an alkaline aqueous solution.

22. The purification method according to claim 16, wherein the fluorine-containing monomer (M) is at least one compound selected from the group consisting of a compound represented by the following formula (M1-1), a compound represented by the following formula (M2-1), a compound represented by the following formula (M2-2), a compound represented by the following formula (M3-1), and a compound represented by the following formula (M3-2):

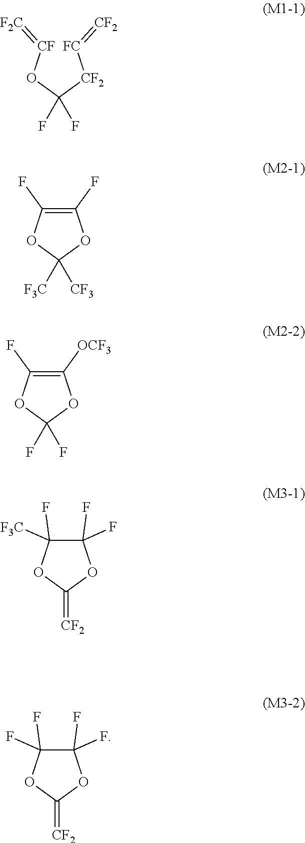

23. The purification method according to claim 16, wherein the fluorine-containing monomer (M) is a compound represented by the following formula (M3-1):

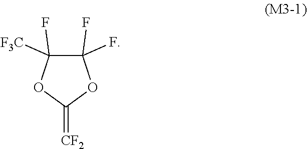

24. The purification method according to claim 23, wherein the fluorine-containing monomer (M) contaminated with fluoride ions is a fluorine-containing monomer contaminated with fluoride ions and a compound represented by the following formula (C):

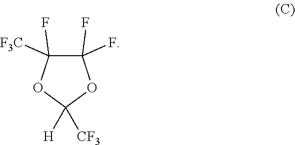 [SMILES: FC(F)(F)C1OC(F)(F)C(F)(C(F)(F)F)O1]

25. The purification method according to claim 24, wherein the content of the compound represented by formula (C) in the fluorine-containing monomer (M) purified is 100 to 100000 ppm by mass.

26. The purification method according to claim 16, wherein the fluorine-containing monomer (M) is a compound represented by the following formula (M3-1): 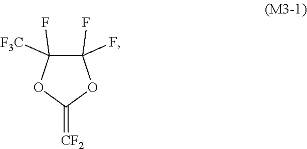
the fluorine-containing monomer (M) contaminated with fluoride ions is a fluorine-containing monomer contaminated with fluoride ions and a compound represented by the following formula (C): 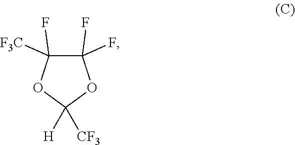 [SMILES: FC(F)(F)C1OC(F)(F)C(F)(C(F)(F)F)O1]
the treatment of the fluorine-containing monomer (M) contaminated with fluoride ions comprises (1) subjecting the fluorine-containing monomer (M) contaminated with fluoride ions to activated carbon adsorption treatment, (2) distilling the fluorine-containing monomer (M) contaminated with fluoride ions and subjecting the distilled fluorine-containing monomer (M) to activated carbon adsorption treatment, or (3) washing the fluorine-containing monomer (M) contaminated with fluoride ions with water or an alkaline aqueous solution,
the fluoride ion content of a fluorine-containing monomer (M) purified is 50 to 600 ppm by mass, and
the content of the compound represented by formula (C) in the fluorine-containing monomer (M) purified is 100 to 100000 ppm by mass.
